(19) United States
(12) Patent Application Publication
(10) Pub. No.: US 2016/0084823 A1
(43) Pub. Date: Mar. 24, 2016

(54) METHODS AND KITS FOR TESTING THE GENOTOXICITY OF AN AGENT

(71) Applicants: INSERM (INSTITUT NATIONAL DE LA SANTÉ ET DE LA RECHERCHE MÉDICALE, Paris (FR); UNIVERSITÉ DE ROUEN, Mont-Saint-Aignan (FR); CENTRE HOSPITALIER UNIVERSITAIRE DE ROUEN, Rouen (FR)

(72) Inventors: Jean-Michel FLAMAN, Rouen (FR); Thierry FREBOURG, Rouen (FR); Gaelle BOUGEARD, Rouen (FR); Yasmine ZERDOUMI, Rouen (FR)

(21) Appl. No.: 14/785,489

(22) PCT Filed: Apr. 17, 2014
(86) PCT No.: PCT/EP2014/057897

§ 371 Date: Oct. 19, 2015

(30) Foreign Application Priority Data
Apr. 19, 2013 (EP) 13305515.2

(51) Int. Cl. G01N 33/50

(52) U.S. Cl. CPC G01N 33/5023

(57) ABSTRACT

The present invention relates to methods and kits for testing the genotoxicity of an agent. In particular, the present invention provides a global assay for the measurement of genotoxic stress based on p53 target gene induction. In particular, the present invention relates to a method for testing the genotoxicity of an agent comprising the steps consisting of i) providing a population of test cells, ii) exposing the populations of test cells with the agent to be tested, iii) determining the expression level of at least one p53 target gene selected from the group consisting of ATF3, BBC3, CABYR, C10ORF10; EMX1; FHL2, GLS2; GRHL3; HES2; IGF-BP4; KIAA0284; PODXL1; RRAD; TP53I3, and XPC in the population of test cells exposed to the agent to be tested iv) comparing the expression level determined at step iii) with the expression level determined when the population of test cells was not exposed to the agent to be tested, and v) concluding that the agent is genotoxic when the expression level determined at step iii) is higher than the level determined when the population of test cells was not exposed to the agent to be tested.

### FIELD OF THE INVENTION

The present invention provides a global assay for the measurement of genotoxic stress based on p53 target gene induction.

### BACKGROUND OF THE INVENTION

The EU legislation REACH (Regulation, Evaluation, Authorization and restriction of CHemicals) foresees the safety assessment of thousands of chemicals within the next decade. Even if animal testing should be undertaken as the last resort, the evaluation of the genotoxic/mutagenic potential appears to be among the end points for which the highest number of in vivo tests will be needed. Over the past 20 years, there have been considerable efforts to develop in vitro or ex vivo methodologies, which can replace experimental animal assays in the identification of potential human mutagens and carcinogens. Typically, the mammalian cellular response to genotoxic damage is often analyzed using a battery of tests, which often include in vitro chromosomal aberration or micronuclei formation tests. Both of these tests visualize DNA damage in cells after exposure to potential genotoxicants by analyzing harvested chromosomes for aberrations (S. M. Galloway, Environ Mol Mutagen (2000) 35:191-201) or by examining micronuclei formed in cells whose DNA has been damaged (W. von der Hude et al., Mutation Res (2000) 468:137-63). However, there are significant problems with interpreting the results of the currently used in vitro genotoxic tests. False positive results in these tests are not uncommon, and the subsequent analysis can be costly and time consuming. Accordingly, assays which can better predict the genotoxic potential of an agent are needed.

The p53 tumor suppressor is a universal sensor of genotoxic stress that regulates the transcription of genes required for appropriate cellular response to DNA damage. In response to DNA damage, the p53 protein is phosphorylated and becomes stabilized upon disruption of an interaction with its negative regulator, MDM2. Subsequent phosphorylation and acetylation of p53 promote different interactions with other proteins allowing its transcriptional activity on target gene regulatory elements to facilitate cell-cycle arrest, apoptosis, or adaptation in response to DNA damage. The frequency of observed mutations in TP53 predicts that its inactivation is a requisite step in tumorigenesis, as p53 is mutated in approximately 50% of human tumors, and as p53 germline mutations are responsible for the Li-Fraumeni syndrome (LFS), a hereditary predisposition to a large spectrum of tumors. Therefore transcriptional induction of p53 target genes can be considered as a global indicator of genotoxic stress. However a global assay for the measurement of genotoxic stress based on p53 target gene induction is still missing.

### SUMMARY OF THE INVENTION

The present invention provides a global assay for the measurement of genotoxic stress in a cell. More particularly the assay is based on the qualitative and quantitative measurement of the transcriptional induction of p53 target genes in 2 populations of cells exposed to the agent to be tested. The two populations of cells are: a population of test cells which carry two copies of a wild type TP53 gene and a population of control cells which harbours at least one p53 dominant negative mutation altering the p53 response to DNA damage. Indeed the use of the population of control cells harboring heterozygous germline TP53 deleterious mutation allows certifying the specificity of the p53 pathway response.

### Detailed Description of the Invention

The present invention relates to a method for testing the genotoxicity of an agent comprising the steps consisting of i) providing a population of test cells which carry two copies of a wild type TP53 gene and a population of control cells which harbours at least one p53 dominant negative mutation altering the p53 response to DNA damage, ii) exposing the populations of cells with the agent to be tested, iii) determining in the exposed population of cells the expression level of at least one p53 target gene selected from the group consisting of ATF3, BBC3, CABYR, C10ORF10; EMX1; FHL2, GLS2; GRHL3; HES2; IGFBP4; KIAA0284; PODXL1; RRAD; TP53I3, and XPC iv) comparing the expression level determined for the population of test cells with the expression level determined for the population of control cells, and v) concluding that the agent is genotoxic when the expression level determined for the population of test cells is higher than the expression level determined for the population of control cells.

As used herein, the term “p53” has its general meaning in the art and refers the tumor suppressor protein containing transcriptional activation, DNA binding, and oligomerization domains. The p53 protein responds to diverse cellular stresses to regulate expression of target genes, thereby inducing cell cycle arrest, apoptosis, senescence, DNA repair, or changes in metabolism. Mutations in this gene are associated with a variety of human cancers, including hereditary cancers such as Li-Fraumeni syndrome.

The term “wild type TP53 gene” refers to the gene encoding p53 protein which does not harbour a p53 dominant negative mutation.

As used the term “p53 dominant negative mutation” has its general meaning in the art and refers to any mutation which results in to a dysfunction of the protein leading to the loss of its transcriptional activity associated with a negative effect on the wild type protein in heterozygous status. p53 loss of function mutations have fully been exemplified in the prior art and thus the skilled man in the art can easily identifies p53 dominant negative mutations (Petitjean A, Mathe E, Kato S, Ishioka C, Tavtigian S V, Hainaut P, Olivier M. Impact of mutant p53 functional properties on TP53 mutation patterns and tumor phenotype: lessons from recent developments in the IARC TP53 database.Hum Mutat. 2007 June; 28(6):622-9) (http://p53.iarc.fr/). p53 dominant negative mutations include somatic and germline mutations indentified in previous studies are mainly missense mutations. Examples of p53 dominant negative mutations include but are not limited to p.R175H, p.R248W and p.R273H.

The term “test cell” refers to any cell that can be used in an in vitro assay providing that the test cell carries two copies of a wild type TP53 gene. Thus the test cell is not isolated from a cell line, since most of the cell lines have a defective p53 system. Besides, the test cell is not a malignant cell.

In a preferred embodiment, the test cell is a human test cell.

In one embodiment, the test cells may be selected from the group consisting of white blood cells than can be easily used in soluble solutions. More particularly, the test cell is a lymphocyte (i.e. a B cell or a T cell). In particular, the population of the test cells is prepared from a PBMC Sample. The term “PBMC” or “peripheral blood mononuclear cells” or “un-fractionated PBMC”, as used herein, refers to whole PBMC, i.e. to a population of white blood cells having a round nucleus, which has not been enriched in a given sub-population. Typically, the PBMC sample according to the invention has not been subjected to a selection step to contain only adherent PBMC (which consist essentially of >90% monocytes) or non-adherent PBMC (which contain T cells, B cells, natural killer (NK) cells, NK T cells and DC precursors). A PBMC sample according to the invention therefore contains lymphocytes (B cells, T cells, NK cells, NKT cells), monocytes, and precursors thereof. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis which will preferentially lyse red blood cells. Such procedures are known to the expert in the art.

In one embodiment, the test cell has been immortalized. As a result said test cell will constitute a cell line that proliferate indefinitely in culture. Methods for immortalizing cells are well known in the art. By a way of example, Epstein Barr virus (“EBV”) can immortalize human lymphocyte.

In one embodiment, the test cell is an EBV-immortalized lymphocyte, in particular a human EBV-immortalized lymphocyte.

The term “control cell” refers to a cell of the same nature than the test cell (e.g. an EBV-immortalized lymphocyte), provided that said cell harbours at least one p53 dominant negative mutation altering the p53 response to DNA damage. Accordingly, the p53 system is defective in the control cell. In one embodiment, the control cells harbour one mutation selected from the group consisting of p.R175H, p.R248W and p.R273H in p53 protein. In one embodiment, the control cells harbour the p.R175H mutation.

Typically, the population of test or control cells may be contained in any appropriate container, such as a plate, dish, flask, tube or cylinder. The container is made of a metal, plastic or glass material. The container comprises any culture medium suitable for the maintenance and viability of the population of cells. Typically, said culture medium may be selected from any commercially available source such as RPMI 1640 medium (GIBCO, Invitrogen). The population of cells are also maintained in appropriate culture conditions. Typically, the population of cells is culture at 37° C. with 5% CO2.

All the genes pertaining to the invention are known per se and sequences of them are publicly available from the GenBank. The genes of the invention are listed in Table A and are described as follows:

1. ATF3: Cyclic AMP-dependent transcription factor ATF-3, Gene ID: 467;

2. BBC3: BCL2 binding component 3, Gene ID: 27113;

3. CABYR: calcium binding tyrosine-(Y)-phosphorylation regulated, Gene ID: 26256;

4. C10ORF10: chromosome 10 open reading frame 10, Gene ID: 11067;

5. EMX1: empty spiracles homeobox 1, Gene ID: 2016;

6. FHL2: four and a half LIM domains 2, Gene ID: 2274;

7. GLS2: glutaminase 2 (liver, mitochondrial), Gene ID: 27165;

8. GRHL3: grainyhead-like 3 (Drosophila), Gene ID: 57822;

9. HES2: hairy and enhancer of split 2 (Drosophila); Gene ID: 54626;

10. IGFBP4: insulin-like growth factor binding protein 4; Gene ID: 3487;

11. KIAA0284: CEP170B: centrosomal protein 1708, Gene ID: 283638;

12. PODXL1: PODXL: podocalyxin-like, Gene ID: 5420;

13. RRAD: Ras-related associated with diabetes, Gene ID: 6236;

14. TP5313: tumor protein p53 inducible protein 3, Gene ID: 9540;

15. XPC: xeroderma pigmentosum, complementation group C, Gene ID: 7508;

In one embodiment, the level of at least 2, 3, 4, 5, 6, 7, 8, 9 or 10 genes is determined at step iii).

In one embodiment, the level of C10ORF10, KIAA0284, RRAD, GLS2, and TP53I3 is determined simultaneously at step iii).

In one embodiment, the level of KIAA0284; PODXL1; RRAD; FHL2; and TP53I3 is determined simultaneously at step iii).

Determining an expression level of a gene in a population of cells can be implemented by a panel of techniques well known in the art.

Typically, an expression level of a gene is assessed by determining the quantity of mRNA produced by this gene.

Methods for determining a quantity of mRNA are well known in the art. For example nucleic acid contained in the population of test cells is first extracted according to standard methods, for example using lytic enzymes or chemical solutions or extracted by nucleic-acid-binding resins following the manufacturer's instructions. The thus extracted mRNA level is then detected by hybridization (e. g., Northern blot analysis, Microarray) and/or PCR amplification (e.g., RT-PCR). Other methods of Amplification include ligase chain reaction (LCR), transcription-mediated amplification (TMA), strand displacement amplification (SDA) and nucleic acid sequence based amplification (NASBA), quantitative new generation sequencing of RNA (NGS). Preferably quantitative or semi-quantitative RT-PCR is preferred. Real-time quantitative or semi-quantitative RT-PCR is particularly advantageous.

In one embodiment, the couple of primers depicted in Table A are used.

In a particular embodiment, the methods of the invention comprise the steps of providing total RNAs extracted from test cells and subjecting the RNAs to amplification and hybridization to specific probes, more particularly by means of a quantitative or semi-quantitative RT-PCR.

In one embodiment, when the determination of the expression levels of several genes is required, multiplex assay may be suitable. In a particular embodiment a RT-QMPSF assay may be used such as described in WO2004009846 and Vezain M, Saugier-Veber P, Melki J, Toutain A, Bieth E, Husson M, Pedespan J M, Viollet L, Pénisson-Besnier I, Fehrenbach S, Bou J, Frébourg T, et al. 2007. A sensitive assay for measuring SMN mRNA levels in peripheral blood and in muscle samples of patients affected with spinal muscular atrophy. Eur J Hum Genet 15:1054-1062.). Briefly, RT-QMPSF is based on PCR amplification of short cDNA sequences (100 to 260 bp), encompassing at least two exons, in a single tube, using dye-labeled primers. RT-QMPSF also included 2 control amplicons for normalization. Fluorescent amplicons are separated on an ABI Prism 3100 Genetic Analyzer (Applied Biosystems, Foster City, Calif.), and the resulting fluorescent profiles are analyzed using the GeneScan 3.7 software (Applied Biosystems). For comparative analyses, RT-QMPSF profiles are superimposed after adjustment of control amplicons to the same heights.

The expression level of a gene in response to an agent is expressed in fold induction compared to not treated cells after normalized expression level on control pics. The expression level of a gene is preferably expressed as normalized expression level when quantitative PCR is used as method of assessment of the expression level because small differences at the beginning of an experiment could provide huge differences after a number of cycles.

Typically, expression levels are normalized by correcting the absolute expression level of a gene by comparing its expression to the expression of a gene that is not relevant for determining the cancer stage of the patient, e.g., a housekeeping gene that is constitutively expressed. Suitable genes for normalization could be housekeeping genes such as the actin gene ACTB, or ribosomal 18S gene. In human immortalized lymphocytes two control genes, SF3A1 and TBP, have been selected for their expression stability in response to stress conditions by transcriptome analysis. This normalization allows comparing the expression level of one sample, e.g., a patient sample, with the expression level of another sample, or comparing samples from different sources.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210)

Other methods for determining the expression level of said genes include the determination of the quantity of proteins encoded by said genes.

Such methods comprise contacting a biological sample with a binding partner capable of selectively interacting with a marker protein present in the sample. The binding partner is generally an antibody, that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate (s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

It is concluded that the agent to be tested is genotoxic when the expression level determined for the exposed population of test cells is at least 2, 3, 4, 5, 6, 7, 8, 9, 10, 11, 12, 13, 14, 15, 16, 17, 18, 19, 20, 21, 22, 23, 24 or 25 fold higher than the level determined for the population of control cells.

In one embodiment, the expression level determined for the exposed populations of cells is compared to the expression level determined when the populations of cells are not exposed to the agent to be tested. Typically, it is concluded that the agent to be tested is genotoxic when i) the expression level determined for the exposed population of test cells is at least 2, 3, 4, 5, 6, 7, 8, 9, 10, 11, 12, 13, 14, 15, 16, 17, 18, 19, 20, 21, 22, 23, 24 or 25 fold higher than the level determined for the population of test cells that was not exposed to the agent to be tested and ii) when the expression level determined for the exposed population of control cells is above the same as the level determined for the population of control cells that was not exposed to the agent to be tested. As used herein the term “above the same” means than the expression level determined for the exposed population of control cells is the same or is higher with a fold lower than 2 than the level determined when the control cells was not exposed to the agent to be tested.

As used herein, the term “agent” refers to any entity for which the genotoxicity can be. tested. Such agent may be a physical agent such as radiations or may be a chemical agent (e.g. a chemical compound). The skilled man in the art can easily select the appropriate method for exposing the population of cells with the agent. For example, the agent (e.g. chemical compounds) may be added to the culture medium of the cells. Alternatively, a container comprising the cells may be exposed to the physical agent (e.g. radiations). The population of cells is exposed to the agent for a time sufficient to induce the activation of p53. Typically, the population of cells could be exposed during different time comprised between 1 to 16 hours.

The method of the invention may be used for very different applications.

For example, the method of the invention is useful for evaluating the genotoxicity of substances other than small organic compounds, and thus may be used to test, for example, radiation; organic extract from environmental samples (for example of polluted air, water, or soil); viruses or other microorganisms; proteins, polynucleotides, polymers and other macromolecules; and the like. The test substance may be obtained from libraries of synthetic or natural compounds, from natural substances, or from organic extracts. In a particular embodiment, the agent is a drug (i.e. a compound that is useful for treating a disease).

For example, the method of the invention is applied for assessing genotoxic properties of novel cosmetics, since in the EU, for cosmetic ingredients, animal testing is generally prohibited since 2009 (EC Regulation 1223/2009).

Environmental pollutants and the like can also be identified using the method of the invention, in which case such compounds are typically identified for further study into their toxic properties. In this application of the method of the invention, one can fractionate an environmental sample (for example, soil, water, or air, suspected of contamination) by known methods (for example chromatography), and subject said fractions to the method of the invention. Fractions that display signs of genotoxicity can then be further fractionated, and (using the method of the invention), the responsible toxic agents identified. Alternatively, one can perform the method of the invention using pure or purified compounds that are suspected of being environmental pollutants to determine their potential for genotoxicity.

The method of the invention is also suitable for screening drug candidate. Candidate drugs that are considered genotoxic by the method of the present invention may be rejected or otherwise dropped from further development. In the case of high-throughput screening applications, such compounds can be flagged as toxic (for example, by the software managing the system in the case of an automated high-throughput system), thus enabling earlier decision making Thus, one can use the method of the invention to prioritize and select candidate compounds for pharmaceutical development based in part on the potential of the compound for genotoxicity. For example, if one has prepared a plurality of compounds (e.g., 50 or more), having similar activity against a selected target, and desires to prioritize or select a subset of said compounds for further development, one can test the entire group of compounds in the method of the invention and discard or reject all those compounds that exhibit positive signs of genotoxicity. This reduces the cost of pharmaceutical development, and the amount invested in any compound selected for development by identifying an important source of toxicity early on.

The method of the invention is also particularly suitable for assessing the capability of a subject to respond to a genotoxic stress. In said embodiment, the population of test cells is prepared from a sample obtained from the subject. Typically a PBMC sample as described above may be prepared from the subject. Then the population of test cells is exposed to an agent which is known to induce genotoxic stress (e.g. a chemotherapeutic agent such as doxorubicin). The method may also be repeated with a plurality of agents that are known to induce gentoxic stress so that the whole capability of the subject to respond to a genotoxic stress may be assessed. It is concluded that the subject has a reduced capability to respond a genotoxic stress when the expression level determined for the population of test cells is above the same than the expression level determined for the control cells. When it is concluded that the subject has a reduced capability to respond to a genotoxic stress, further clinical investigations may be carried out to understand why the subject has this low capability. For example, mutations in TP53 gene may be researched. Thus the method of the invention represents a quick test for determining whether a subject has a defective p53 pathway response. The method of the invention is also particularly suitable of determining whether the subject is eligible for a treatment with a drug that is known to induce genotoxic stress. The method of the invention is also suitable for monitoring the treatment of a subject with a drug that is known to induce genotoxic stress.

The method of the invention is also suitable for screening a drug useful for reducing the genotoxic stress in a subject in need thereof. Typically, the method comprises the steps consisting of i) providing a population of test cells which carry two copies of a wild type TP53 gene and a population of control cells which harbours at least one p53 dominant negative mutation altering the p53 response to DNA damage, ii) exposing the populations of cells with an agent that is known to induce genotoxic stress, iii) bringing the population of cells into contact with candidate compound to be tested, iii) determining in the exposed population of cells the expression level of at least one p53 target gene selected from the group consisting of ATF3, BBC3, CABYR, C10ORF10; EMX1; FHL2, GLS2; GRHL3; HES2; IGFBP4; KIAA0284; PODXL1; RRAD; TP53I3, and XPC iv) comparing the expression level determined for the population of test cells with the expression level determined for the population of control cells, and v) positively selected the candidate compound when the expression level determined for the population of test cells is above than the expression level determined for the population of control cells.

A further object of the invention relates to kits for implementing of the invention, wherein said kits comprise means for measuring the expression level of at least one gene selected from table A of the invention in the sample obtained from the patient. Typically, the kit may also comprise the population of test cells and also may comprise the population of control cells. The kit may also comprise all the means for preparing the population of test cells (e.g. from a PBMC sample obtained from the subject), a sample of control cells, all the means for culturing the population of cells and all the means for analysing the expression of the selected genes. The kit of the invention typically includes probes, primers macroarrays or microarrays as above described. For example, the kit may comprise a set of probes as above defined, usually made of DNA, and that may be pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards. Alternatively the kit of the invention may comprise amplification primers that may be pre-labelled or may contain an affinity purification or attachment moiety. The kit may further comprise amplification reagents and also other suitably packaged reagents and materials needed for the particular amplification protocol. In one embodiment, the kit comprises at least one couple of primers depicted in Table A.

In one embodiment, the kit comprises the primer SEQ ID NO:1 and the primer SEQ ID NO:2.

In one embodiment, the kit comprises the primer SEQ ID NO:3 and the primer SEQ ID NO:4.

In one embodiment, the kit comprises the primer SEQ ID NO:5 and the primer SEQ ID NO:6.

In one embodiment, the kit comprises the primer SEQ ID NO:7 and the primer SEQ ID NO:8.

In one embodiment, the kit comprises the primer SEQ ID NO:9 and the primer SEQ ID NO:10.

In one embodiment, the kit comprises the primer SEQ ID NO:11 and the primer SEQ ID NO:12.

In one embodiment, the kit comprises the primer SEQ ID NO:13 and the primer SEQ ID NO:14.

In one embodiment, the kit comprises the primer SEQ ID NO:15 and the primer SEQ ID NO:16.

In one embodiment, the kit comprises the primer SEQ ID NO:17 and the primer SEQ ID NO:18.

In one embodiment, the kit comprises the primer SEQ ID NO:19 and the primer SEQ ID NO:20.

In one embodiment, the kit comprises the primer SEQ ID NO:21 and the primer SEQ ID NO:22.

In one embodiment, the kit comprises the primer SEQ ID NO:23 and the primer SEQ ID NO:24.

In one embodiment, the kit comprises the primer SEQ ID NO:25 and the primer SEQ ID NO:26.

In one embodiment, the kit comprises the primer SEQ ID NO:27 and the primer SEQ ID NO:28.

In one embodiment, the kit comprises the primer SEQ ID NO:29 and the primer SEQ ID NO:30.

The invention will be further illustrated by the following figures and examples. However, these examples and figures should not be interpreted in any way as limiting the scope of the present invention.

### FIGURES

FIG. 1: p53 pathway in response to DNA damage.

FIG. 2: p53 pathway in response to DNA damage. A RT-QMPSF analysis of control lymphocytes treated or not by doxorubicin. B. RT-QMPSF analysis of patients lymphocytes with 175H TP53 mutation treated or not by doxorubicin. Arrows indicate the level of expression in untreated cell. Profiles have been aligned on the 2 control amplicons (SF3A1 and TBP).

FIG. 3: p53 target genes strongly and specifically induced in control lymphocytes (T401) exposed to DNA damage. Experiments have been performed in triplicates and standard deviation is indicated.

FIG. 4: Validation of the new genotoxicity assay based on p53 pathway activation detection using positive and negative mutagen compounds. Known mutagens are indicated in bold.

### EXAMPLE 1

### A Global Assay for the Measurement of Genotoxic Stress Based on P53 Target Gene Induction and TP53 MT/WT LYMPHOCYTES AS SENSOR CELLS

The EU legislation REACH (Regulation, Evaluation, Authorization and restriction of CHemicals) foresees the safety assessment of thousands of chemicals within the next decade. Even if animal testing should be undertaken as the last resort, the evaluation of the genotoxic/mutagenic potential appears to be among the end points for which the highest number of in vivo tests will be needed. Over the past 20 years, there have been considerable efforts to develop in vitro or ex vivo methodologies, which can replace experimental animal assays in the identification of potential human mutagens and carcinogens.

The ability of the in vitro bacterial and mammalian cell tests currently used to identify genotoxic molecules has been shown to be limited by a high rate of false-positive results. These assays test molecules at high concentrations which has been identified as one possible source of false positives. Thus alternative ex vivo or in vitro methods for safety evaluation of chemicals are urgently needed.

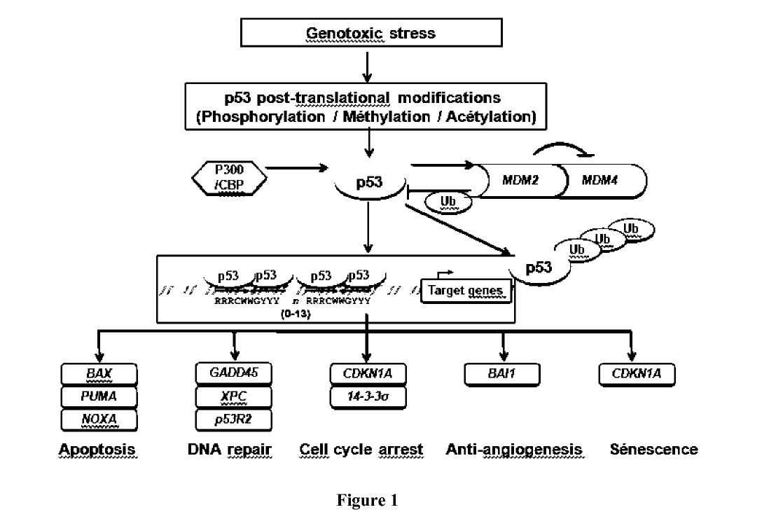

The p53 tumor suppressor is a universal sensor of genotoxic stress that regulates the transcription of genes required for appropriate cellular response to DNA damage (FIG. 1). In response to DNA damage, the p53 protein is phosphorylated and becomes stabilized upon disruption of an interaction with its negative regulator, MDM2. Subsequent phosphorylation and acetylation of p53 promote different interactions with other proteins allowing its transcriptional activity on target gene regulatory elements to facilitate cell-cycle arrest, apoptosis, or adaptation in response to DNA damage. The frequency of observed mutations in TP53 predicts that its inactivation is a requisite step in tumorigenesis, as p53 is mutated in approximately 50% of human tumors, and as p53 germline mutations are responsible for hereditary predisposition to a large spectrum of tumors. Therefore transcriptional induction of p53 target genes can be considered as a global indicator of genotoxic stress.

P53 Genotoxicity Assay Principle

Then, we propose a new ex-vivo genotoxicity assay based on the qualitative and quantitative measurement of the transcriptional induction of p53 target genes after exposure of human lymphocytes to chemical or physical agents. The use of lymphocytes derived from LFS patients harboring heterozygous germline TP53 deleterious mutation allows to certify the specificity of the p53 pathway response.

In the first version of this assay EBV-immortalized human lymphocytes derived from healthy blood donor with wild type TP53 or Li-Fraumeni patients harboring germline heterozygous TP53 mutations are cultured in 24 well plates, during 5 h, with or without the chemicals to test. RNA is then extracted using Nucleospin RNAII kit (Macherey Nagel). 50 ng of total RNA is used to perform RT-QMPSF on p53 target genes selected to be strongly and specifically induced in response to DNA damage. The selection of these target genes has been previously performed from data generated with Agilent whole human genome expression microarrays, on control and LFS patients lymphocytes treated or not with Doxorubicin. RT-QMPSF analysis using 5 TP53 target genes and 2 control amplicons are performed on cells treated or not with the chemicals and profiles were compared after normalization on controls amplicons. Genotoxic activity is detected when more than two target genes showed 2 fold induction.

The different advantages of this new genotoxicity assay are:
The use of the p53 pathway activation, as a universal signal indicative of a genotoxic stress and DNA damages.
The use on non malignant cells which are representative of the genetic background of patients.
The specificity of the assay ensured by the use, as controls, of TP53 wt/mt lymphocytes.
The sensitivity of the assay with genotoxic activity detected below the micromolar.
The targeted analysis of selected genes using RT-QMPSF which is a simple, rapid and robust technique.
The possibility to use the assay not only to test chemical or physical agents for their genotoxic activity but also to test molecules which could reduce gentoxic stress.

Proof of Concept

Proof of concept of the new genotoxicity assay has been obtained in human lymphocytes derived from 3 healthy control subjects and TP53 mutation carriers with missense mutations (p.R175H; p.R248W; p.R273H). The lymphocytes have been treated with doxorubicin, a well characterized mutagen chemical inhibiting topoisomerase II, for 5 hours and the different transcriptomes have been compared to identify p53 target genes, strongly and specifically in control subjects, induced in response to DNA damage.

EBV-Transformed Lymphocytes Culture and Treatment

Peripheral blood lymphocytes from healthy control subjects and TP53 mutation carriers with missense mutations (p.R175H; p.R248W; p.R273H) were immortalized by Epstein-Barr Virus (EBV) infection (B95.8 strain). In each case, the TP53 genotype of EBV-immortalized lymphocytes was checked by sequencing and QMPSF analyses. EBV-transformed lymphocytes were maintained in RPMI 1640 medium (GIBCO, Invitrogen), supplemented with 10% heat-inactivated fetal bovine serum (Invitrogen) at 37° C. with 5% CO₂. Cells were seeded in duplicates in 24-well plates (Corning) at a density of 5.10⁵ cells/well, and treated or not with 200 ng/mL of doxorubicin (Sigma-Aldrich) for 5 h, harvested by centrifugation, and then protein and RNA were extracted.

Validation of p53 Pathway Activation by Western Blot Analysis

After DNA damage, p53 pathway activation was checked by western blot analysis of p53 protein accumulation. In brief, cells were pelleted and homogenized in RIPA buffer (25 mM Tris-HCl, pH 7.6, 150 mM NaCl, 1% NP-40, 1% sodium deoxycholate, and 0.1% SDS), supplemented with cocktails of protease inhibitors (Sigma-Aldrich) and phosphatase inhibitors (Pierce Biotechnology). Samples were centrifuged at 11300 g for 20 min at 4° C. to and the supernatant was collected as the protein extract. Thirty μg of protein were resolved by a 10% SDS-PAGE. Proteins were transferred to nitrocellulose membranes (Hybond CExtra; Amersham Biosciences, Arlington Heights, Ill.). The following primary antibodies were used: mouse monoclonal anti-p53 DO-1 antibody (1/2000; Santa Cruz Biotechnology, Inc.), anti-actin JLA20 antibody (1/10000; Sigma Aldrich). Membranes were incubated with peroxidase-labeled anti-mouse or anti-rabbit antibodies (1/10000) from Jackson Immunoresearch Laboratories (West Grove, Pa.), and signals were detected with chemiluminescence reagents using G:Box (Syngene) and GeneSnap software.

RNA Extraction for Comparative Gene Expression Profiling

After treatment cells were centrifuged, lysed using RA1 reagent from Nucleospin RNAII (Macherey Nagel), RNA was extracted following the manufacturer's instructions and quantified using a ND-1000 UV-Vis Spectrophotometer (NanoDrop technologies). The integrity of the RNA was assessed with the Agilent 2100 Bioanalyzer (Agilent), according to the manufacturer's instructions. RNA samples used in this study all had a 260/280 ratio above 1.9 and a RNA Integrity Number (RIN) above 9.0.

Comparative Gene Expression Profiling for p53 Target Genes Selection

Comparative gene expression profiling of lymphocytes treated or not with doxorubicin was performed using Whole Human Genome Oligo 4×44K Microarray (G4112F, Agilent), according to the Agilent Two-Color Gene Expression workflow. Briefly, total RNA (100 ng) was reverse transcribed into cDNA, cRNA was subsequently synthesized and labeled using the low input Quick Amp Labeling Kit (Agilent), with Cy3 (for untreated cells) and Cy5 (for doxorubicin treated cells), and purified using the RNeasy mini kit (Qiagen). For each EBV-immortalized lymphocyte sample, 825 ng of cRNA derived from untreated and doxorubicin treated cells were co-hybridized for 17 h at 65° C. in an Agilent's microarray hybridization rotator oven. Fluorescence signals of the hybridized microarrays were detected using an Agilent's DNA microarray scanner G2565CA (Agilent Technologies) with a resolution of 5 μm. For each EBV-immortalized lymphocyte sample, comparative gene expression profiling was performed on 2 independent pairs of treated and untreated samples.

Image analysis and extraction of raw and corrected signal intensities were performed with the Feature Extraction Software 10.5.1.1 (Agilent Technologies). Data were normalized using the GeneSpring GX 10.0.2 software (Agilent Technologies) and the recommended two color analysis workflow. Differentially expressed genes between doxorubicin treated and untreated cells were arbitrarily defined using, as filters, a p value<0.01 and fold change cutoffs> or <2, for up and down regulation, respectively.

Detection of p53 Pathway Activation by RT-QMPSF Analysis

RT-QMPSF is based on PCR amplification of short cDNA sequences (100 to 260 bp), encompassing at least two exons, in a single tube, using dye-labeled primers. RT-QMPSF also included 2 control amplicons for normalization. Fluorescent amplicons were separated on an ABI Prism 3100 Genetic Analyzer (Applied Biosystems, Foster City, Calif.), and the resulting fluorescent profiles were analyzed using the GeneScan 3.7 software (Applied Biosystems). For comparative analyses, RT-QMPSF profiles were superimposed after adjustment of control amplicons to the same heights. All QMPSF analyses were performed at least in duplicates.

Preliminary Results

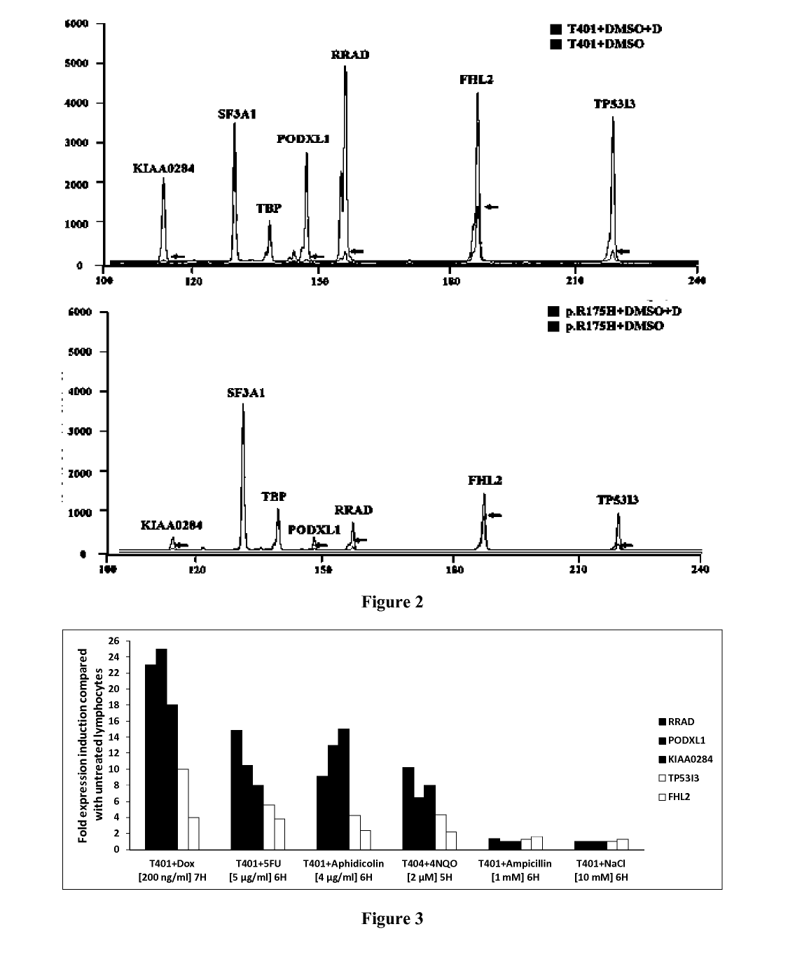

Lymphocytes derived from a healthy control and from a Li-Fraumeni patient with p.R175H germline mutations have been used to test the assay with 0.3 μM doxorubicin. As show in FIG. 2, RT-QMPSF analysis showed clearly expression induction of 5 p53 target genes (KIAA0284; PODXL1; RRAD; FHL2; TP53I3) specifically in control lymphocytes.

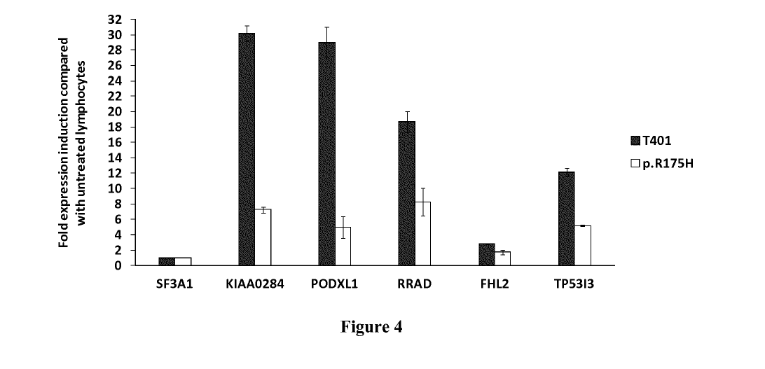

The p53 genotoxicity assay for doxorubicin has been performed in triplicates showing that the assay is very robust (FIG. 3). We have tested 5 well characterized mutagens which alter DNA with different mechanism. For all of them the assay was very efficient and sensitive (FIG. 4).

TABLE A
Name, description, primers and functins of the genes according to the invention

| | | | | | Linked | |
|---|---|---|---|---|---|---|
| Name | Description | Primer name | Sequence | Size | to p53 | Proper function |
| C10ORF10 | Chromosome 10 | RT-C10orf10-F | ACTGGCTTTTTG | 114 | No | ? |
| | ORF 10 | | GGGAGTCC | | | |
| | | | (SEQ ID NO: 1) | | | |
| C10ORF10 | Chromosome 10 | RT-C10orf10-R | GCTTGCTGCTG | 114 | No | ? |
| | ORF 10 | | TCCATCTGT | | | |
| | | | (SEQ ID NO: 2) | | | |
| KIAA0284 | Centrosomal | RT-kiaa0284-F | TCAACGCCGAG | 117 | No | Mitosis |
| | protein 170B | | AACGAGG | | | (Cep170B) |
| | | | (SED ID NO: 3) | | | |
| KIAA0284 | Centrosomal | RT-kiaa0284-R | GGTCCACGATG | 117 | No | Mitosis |
| | protein 170B | | GCATTGAT | | | (Cep170B) |
| | | | (SED ID NO: 4) | | | |
| RRAD | Ras-related | RT-RRAD-F | CTCGTGAGGTC | 158 | Yes | Rho Kinases |
| | small | | TCGGTGGAT | | | inhibitor |
| | GTPase (RRAD) | | (SED ID NO: 5) | | | |
| RRAD | Ras-related | RT-RRAD-R | TGCGTTGGCTT | 158 | Yes | Rho Kinases |
| | small | | CTTTGCTGT | | | inhibitor |
| | GTPase (RRAD) | | (SED ID NO: 6) | | | |
| GLS2 | Glutaminase 2 | RT-GLS2-F | AGGACAGGTGG | 200 | Yes | Mitochondrial |
| | | | GGCAACATT | | | enzyme: gluatamate |
| | | | (SED ID NO: 7) | | | synthesis |
| GLS2 | Glutaminase 2 | RT-GLS2-R | GCTTTTTCTTGA | 200 | Yes | Mitochondrial |
| | | | GACAGGGGC | | | enzyme: gluatamate |
| | | | (SED ID NO: 8) | | | synthesis |
| CABYR | Calcium | RT-Cabyr-F | TTCAGGAAGCA | 212 | No | ? |
| | Binding | | CAGGGATGG | | | |
| | tyrosine-(Y)- | | (SED ID NO: 9) | | | |
| | phosphory- | | | | | |
| | lation | | | | | |
| CABYR | Calcium | RT-Cabyr-R | TGGGAAAGCAA | 212 | No | ? |
| | Binding | | CAGAAAGGA | | | |
| | tyrosine-(Y)- | | (SED ID NO: 10) | | | |
| | phosphory- | | | | | |
| | lation | | | | | |
| TP53I3 | P53-Induced | RT-TP53I3-F | ATGGTCTGATG | 220 | Yes | stress |
| | Gene3 | | GGAGGAGGT | | | oxidatif |
| | | | (SED ID NO: 11) | | | |
| TP53I3 | P53-Induced | RT-TP53I3-R | TGGATTTCGGT | 220 | Yes | stress |
| | Gene3 | | CACTGGGTA | | | oxidatif |
| | | | (SED ID NO: 12) | | | |
| PODXL1 | Podocalyxin | RT-Podxl-F | CATCATTCCTG | 150 | Yes | proteine |
| | | | CTCCTCGTG | | | d'adhesion |
| | | | (SED ID NO: 13) | | | |
| PODXL1 | Podocalyxin | RT-Podxl-R | AAGAGGTCTCC | 150 | Yes | proteine |
| | | | ATCACTTCCAG | | | d'adhesion |
| | | | (SED ID NO: 14) | | | |
| ATF3 | Activating | RT-ATF3-F | CTCCTGGGTCA | 211 | Yes | Transcription |
| | Transcription | | CTGGTGTTTG | | | repression |
| | Factor 3 | | (SED ID NO: 15) | | | |
| ATF3 | Activating | RT-ATF3-R | CTTTCTCGTCGC | 211 | Yes | Transcription |
| | Transcription | | CTCTTTTTC | | | repression |
| | Factor 3 | | (SED ID NO: 16) | | | |
| FHL2 | Four and a | RT-FHL2-F | GCGATGACTTT | 188 | Yes | Transcription |
| | Half LIM | | GCCTACTGC | | | repression |
| | domains 2 | | (SED ID NO: 17) | | | |
| FHL2 | Four and a | RT-FHL2-R | TGTGAGGAAGC | 188 | Yes | Transcription |
| | half LIM | | CACGCCC | | | repression |
| | domains 2 | | (SED ID NO: 18) | | | |
| HES2 | Hairy and | RT-HES2-F | GCCCTGCTCAC | 179 | No | Transcription |
| | enhancer of | | CCCACAT | | | repression |
| | Split 2 | | (SED ID NO: 19) | | | |
| | (Drosophila) | | | | | |
| HES2 | Hairy and | RT-HES2-R | AAGGATTTGTT | 179 | No | Transcription |
| | enhancer of | | TTCCCGAGCA | | | repression |
| | Split 2 | | (SED ID NO: 20) | | | |
| | (Drosophila) | | | | | |
| BBC3 | BCL2 binding | RT-PUMA-F | GGACGACCTCA | 105 | Yes | Apoptosis |
| | component 3 | | ACGCACAG | | | |
| | | | (SED ID NO: 21) | | | |
| BBC3 | BCL2 binding | RT-PUMA-R | GGCAGGAGTCC | 105 | Yes | Apoptosis |
| | component 3 | | CATGATGA | | | |
| | | | (SED ID NO: 22) | | | |
| XPC | Xeroderma | RT-XPC-F | AAGCCAGTGGA | 148 | No | DNA repair |
| | Pigmentosum C | | GATAGAGATTG | | | |
| | | | A | | | |
| | | | (SED ID NO: 23) | | | |
| XPC | Xeroderma | RT-XPC-R | GGTGAACCTTG | 148 | No | DNArepair |
| | Pigmentosum C | | TGTGTGTCCTC | | | |
| | | | (SED ID NO: 24) | | | |
| IGFBP4 | Insulin Growth | RT-IGFBP4-F | CATGGAGCTGG | 138 | Yes | Cell |
| | Factor Binding | | CGGAGAT | | | proliferation |
| | Protein | | (SED ID NO: 25) | | | |
| IGFBP4 | Insulin Growth | RT-IGFBP4-R | ATTTTGGCGAA | 138 | Yes | Cell |
| | Factor Binding | | GTGCTTCTG | | | proliferation |
| | Protein | | (SED ID NO: 26) | | | |
| GRHL3 | Grainyhead- | RTGRHL3-F | GGAGATGATGA | 116 | No | Transcription |
| | like 3 | | CAGTGTTGCG | | | factor |
| | | | (SED ID NO: 27) | | | |
| GRHL3 | Grainyhead- | RTGRHL3-R | TACCTCTTTCCT | 116 | No | Transcription |
| | like 3 | | TGGTCATTCC | | | factor |
| | | | (SED ID NO: 28) | | | |
| EMX1 | Empty | RT-EMX1-F | GAGACGCAGGT | 201 | No | Transcription |
| | spiracles | | GAAGGTGTG | | | factor |
| | homeobox 1 | | (SED ID NO: 29) | | | |
| EMX1 | Empty | RT-EMX1-R | CTCGTGGGTTT | 201 | No | Transcription |
| | spiracles | | GTGGTTGC | | | factor |
| | homeobox 1 | | (SED ID NO: 30) | | | |

### REFERENCES

Throughout this application, various references describe the state of the art to which this invention pertains. The disclosures of these references are hereby incorporated by reference into the present disclosure.

1. A method for testing the genotoxicity of an agent comprising the steps of i) providing a population of test cells which carry two copies of a wild type TP53 gene and a population of control cells which harbours at least one p53 dominant negative mutation altering the p53 response to DNA damage, ii) exposing the populations of cells to the agent to be tested, iii) determining in the exposed population of cells an expression level of at least one p53 target gene selected from the group consisting of ATF3, BBC3, CABYR, C10ORF10; EMX1; FHL2, GLS2; GRHL3; HES2; IGFBP4; KIAA0284; PODXL1; RRAD; TP53I3, and XPC iv) comparing the expression level determined for the population of test cells with the expression level determined for the population of control cells, and v) concluding that the agent is genotoxic when the expression level determined for the population of test cells is higher than the expression level determined for the population of control cells.

2. The method of claim 1 wherein the populations of test cells or the population of control cells are human EBV-immortalized lymphocytes.

3. The method of claim 1 wherein the population of control cells harbors the p.R175H mutation.

4. The method according to claim 1 wherein the expression level of at least 2, 3, 4, 5, 6, 7, 8, 9 or 10 genes is determined.

5. The method according to claim 1 wherein expression levels of C10ORF10, KIAA0284, RRAD, GLS2, and TP53I3 are determined simultaneously at step iii).

6. The method according to claim 1 wherein expression levels of KIAA0284; PODXL1; RRAD; FHL2; and TP53I3 is are determined simultaneously at step iii).

7. The method according to claim 1 wherein the expression levels of the genes are determined with a RT-QMPSF assay.

8. The method according to claim 1 wherein it is concluded that the agent to be tested is genotoxic when the expression level determined for the exposed population of test cells is at least 2, 3, 4, 5, 6, 7, 8, 9, 10, 11, 12, 13, 14, 15, 16, 17, 18, 19, 20, 21, 22, 23, 24 or 25 fold higher than the level determined for the population of control cells.

9. The method according to claim 1 wherein the expression level determined for the exposed populations of cells is compared to the expression level determined when the populations of cells is not exposed to the agent to be tested.

10. The method of claim 9 wherein it is concluded that the agent to be tested is genotoxic when i) the expression level determined for the exposed population of test cells is at least 2, 3, 4, 5, 6, 7, 8, 9, 10, 11, 12, 13, 14, 15, 16, 17, 18, 19, 20, 21, 22, 23, 24 or 25 fold higher than the level determined for the population of test cells that was not exposed to the agent to be tested and ii) when the expression level determined for the exposed population of control cells is above as the level determined for the population of control cells that was not exposed to the agent to be tested.

11. The method according to claim 1 wherein the agent is physical agent such as radiation.

12. The method according claim 1 wherein the agent is chemical compound.

13. The method of claim 9 wherein the chemical compound is a drug.

14. A method for screening a drug useful for reducing genotoxic stress in a subject in need thereof comprising the steps of i) providing a population of test cells which carry two copies of a wild type TP53 gene and a population of control cells which harbours at least one p53 dominant negative mutation altering the p53 response to DNA damage, ii) exposing the populations of cells to an agent that is known to induce genotoxic stress, iii) bringing the population of cells into contact with candidate compound to be tested, iii) determining in the exposed population of cells an expression level of at least one p53 target gene selected from the group consisting of ATF3, BBC3, CABYR, C10ORF10; EMX1; FHL2, GLS2; GRHL3; HES2; IGFBP4; KIAA0284; PODXL1; BRAD; TP5313, and XPC iv) comparing the expression level determined for the population of test cells with the expression level determined for the population of control cells, and v) positively selecting the candidate compound when the expression level determined for the population of test cells is above the expression level determined for the population of control cells.

15. A kit comprising at least one pair of primers depicted in table A.

16. The kit of claim 15 comprising a population of control cells.

17. The kit of claim 16 comprising of at least one control cell which harbours a mutation selected from the group consisting of p.R175H, p.R248W and p.R273H in p53 protein.

18. The kit of claim 15 which comprises the primer SEQ ID NO:1 and the primer SEQ ID NO:2.

19. The kit of claim 15 which comprises the primer SEQ ID NO:3 and the primer SEQ ID NO:4.

20. The kit of claim 15 which comprises the primer SEQ ID NO:5 and the primer SEQ ID NO:6.

21. The kit of claim 15 which comprises the primer SEQ ID NO:7 and the primer SEQ ID NO:8.

22. The kit of claim 15 which comprises the primer SEQ ID NO:9 and the primer SEQ ID NO:10.

23. The kit of claim 15 which comprises the primer SEQ ID NO:11 and the primer SEQ ID NO:12.

24. The kit of claim 15 which comprises the primer SEQ ID NO:13 and the primer SEQ ID NO:14.

25. The kit of claim 15 which comprises the primer SEQ ID NO:15 and the primer SEQ ID NO:16.

26. The kit of claim 15 which comprises the primer SEQ ID NO:17 and the primer SEQ ID NO:18.

27. The kit of claim 15 which comprises the primer SEQ ID NO:19 and the primer SEQ ID NO:20.

28. The kit of claim 15 which comprises the primer SEQ ID NO:21 and the primer SEQ ID NO:22.

29. The kit of claim 15 which comprises the primer SEQ ID NO:23 and the primer SEQ ID NO:24.

30. The kit of claim 15 which comprises the primer SEQ ID NO:25 and the primer SEQ ID NO:26.

31. The kit of claim 15 which comprises the primer SEQ ID NO:27 and the primer SEQ ID NO:28.

32. The kit of claim 15 which comprises the primer SEQ ID NO:29 and the primer SEQ ID NO:30.


SEQUENCE LISTING (30 sequences)

SEQ ID NO: 1 - DNA, 20 bp, Artificial
  Synthetic oligonucleotide primer
actggctttttggggagtcc

SEQ ID NO: 2 - DNA, 20 bp, Artificial
  Synthetic oligonucleotide primer
gcttgctgctgtccatctgt

SEQ ID NO: 3 - DNA, 18 bp, Artificial
  Synthetic oligonucleotide primer
tcaacgccgagaacgagg

SEQ ID NO: 4 - DNA, 19 bp, Artificial
  Synthetic oligonucleotide primer
ggtccacgatggcattgat

SEQ ID NO: 5 - DNA, 20 bp, Artificial
  Synthetic oligonucleotide primer
ctcgtgaggtctcggtggat

SEQ ID NO: 6 - DNA, 20 bp, Artificial
  Synthetic oligonucleotide primer
tgcgttggcttctttgctgt

SEQ ID NO: 7 - DNA, 20 bp, Artificial
  Synthetic oligonucleotide primer
aggacaggtggggcaacatt

SEQ ID NO: 8 - DNA, 20 bp, Artificial
  Synthetic oligonucleotide primer
gctttttcttgagcaggggc

SEQ ID NO: 9 - DNA, 20 bp, Artificial
  Synthetic oligonucleotide primer
ttcaggaagcacagggatgg

SEQ ID NO: 10 - DNA, 20 bp, Artificial
  Synthetic oligonucleotide primer
tgggaaagcaacagaaagga

SEQ ID NO: 11 - DNA, 20 bp, Artificial
  Synthetic oligonucleotide primer
atggtctgatgggaggaggt

SEQ ID NO: 12 - DNA, 20 bp, Artificial
  Synthetic oligonucleotide primer
tggatttcggtcactgggta

SEQ ID NO: 13 - DNA, 20 bp, Artificial
  Synthetic oligonucleotide primer
catcattcctgctcctcgtg

SEQ ID NO: 14 - DNA, 22 bp, Artificial
  Synthetic oligonucleotide primer
aagaggtctccatcacttccag

SEQ ID NO: 15 - DNA, 21 bp, Artificial
  Synthetic oligonucleotide primer
ctcctgggtcactggtgtttg

SEQ ID NO: 16 - DNA, 21 bp, Artificial
  Synthetic oligonucleotide primer
ctttctcgtcgcctctttttc

SEQ ID NO: 17 - DNA, 20 bp, Artificial
  Synthetic oligonucleotide primer
gcgatgactttgcctactgc

SEQ ID NO: 18 - DNA, 18 bp, Artificial
  Synthetic oligonucleotide primer
tgtgaggaagccacgccc

SEQ ID NO: 19 - DNA, 18 bp, Artificial
  Synthetic oligonucleotide primer
gccctgctcaccccacat

SEQ ID NO: 20 - DNA, 21 bp, Artificial
  Synthetic oligonucleotide primer
aaggatttgttttcccgagca

SEQ ID NO: 21 - DNA, 19 bp, Artificial
  Synthetic oligonucleotide primer
ggacgacctcaacgcacag

SEQ ID NO: 22 - DNA, 19 bp, Artificial
  Synthetic oligonucleotide primer
ggcaggagtcccatgatga

SEQ ID NO: 23 - DNA, 23 bp, Artificial
  Synthetic oligonucleotide primer
aagccagtggagatagagattga

SEQ ID NO: 24 - DNA, 22 bp, Artificial
  Synthetic oligonucleotide primer
ggtgaaccttgtgtgtgtcctc

SEQ ID NO: 25 - DNA, 18 bp, Artificial
  Synthetic oligonucleotide primer
catggagctggcggagat

SEQ ID NO: 26 - DNA, 20 bp, Artificial
  Synthetic oligonucleotide primer
attttggcgaagtgcttctg

SEQ ID NO: 27 - DNA, 21 bp, Artificial
  Synthetic oligonucleotide primer
ggagatgatgacagtgttgcg

SEQ ID NO: 28 - DNA, 22 bp, Artificial
  Synthetic oligonucleotide primer
tacctctttccttggtcattcc

SEQ ID NO: 29 - DNA, 20 bp, Artificial
  Synthetic oligonucleotide primer
gagacgcaggtgaaggtgtg

SEQ ID NO: 30 - DNA, 19 bp, Artificial
  Synthetic oligonucleotide primer
ctcgtgggtttgtggttgc